(19) United States
(12) Patent Application Publication
(10) Pub. No.: US 2024/0189517 A1
(43) Pub. Date: Jun. 13, 2024

(54) DRUG INJECTION STOPPER WITH THIN FILM LUBRICANT

(71) Applicant: W.L. GORE & ASSOCIATES, INC., Newark, DE (US)

(72) Inventors: Naomi S. DELONG, Newark, DE (US); Peter HEICKSEN, Newark, DE (US)

(21) Appl. No.: 18/039,389

(22) PCT Filed: Dec. 17, 2021
(86) PCT No.: PCT/US21/73016

§ 371 Date: May 30, 2023

Related U.S. Application Data
(60) Provisional application No. 63/131,371, filed on Dec. 29, 2020.

(51) Int. Cl. A61M 5/315

(52) U.S. Cl. CPC A61M 5/31513; A61M 2205/0216; A61M 2205/0222; A61M 2205/0238

(57) ABSTRACT

A syringe stopper containing a low level (i.e., from about 0.3 μg to about 100 μg) of a thin film lubricant is provided. The stopper may be positioned within a barrel of a syringe or an autoinjector. The stopper includes an elastomeric body, a solid lubricant, and a thin film lubricant. The amount of thin film lubricant present on a stopper is below a level that will induce particulation in a silicone free barrel. Additionally, the low level of thin film lubricant allows for a lower breakaway force (both wet and dry), average slide force, and a reduction in glide force compared to stoppers with no thin film lubricant. The low amount of the thin film lubricant present on the stopper also lowers the average insertion force within a syringe or vent tube compared to stoppers with no thin film lubricant, thereby allowing improved machinability inclusive of insertion.

### FIELD

The present disclosure relates generally to an injection device having a low level of lubricant. More specifically, the disclosure relates to injection devices having an elastomeric stopper with a low level of lubricant which interacts with a surface of the stopper and methods for making and using the same.

### BACKGROUND

Injection devices (e.g., syringes, autoinjectors) used for delivery of drugs include a barrel and a stopper. The stopper is slidably fitted within the barrel and may have a plunger rod affixed to it for actuation of the syringe and delivery of a drug. A liquid lubricant (e.g., silicone oil) is often provided in the injection device to reduce sliding friction between the stopper and the barrel and to improve the seal between them. Pre-filled injection devices may be used as a way to both store and deliver drugs. However, the liquid lubricant present in the injection device may diffuse into the drug contained therein and which may be injected into a patient. Silicone oil may be of particular concern with biopharmaceuticals, because it can cause aggregation of certain proteins, thereby rendering the biopharmaceutical unusable for injection.

### SUMMARY

According to a one Aspect (“Aspect 1”), a syringe includes a barrel configured to hold a therapeutic agent and a plunger rod positioned at least partially within the barrel. The plunger rod includes a stopper having an elastomeric body, a solid lubricant on at least a portion of the exterior of the elastomeric body, and a thin film lubricant positioned between the solid lubricant and an interior of the barrel. A mass of the thin film lubricant on the stopper is from about 0.3 μg to about 100 μg.

According to another Aspect (“Aspect 2”) further to Aspect 1, the thin film lubricant is silicone and the solid lubricant is a fluoropolymer.

According to another Aspect (“Aspect 3”), further to Aspect 1 or Aspect 2, the solid lubricant and the thin film lubricant on the stopper represent the total lubricant in the syringe.

According to another Aspect (“Aspect 4”), further to any one of Aspects 1 to 3, the stopper is configured to be slidably moved within the barrel with a dry breakaway force less than about 15N.

According to one Aspect (“Aspect 5”), a syringe includes a barrel configured to hold a therapeutic agent and a plunger rod positioned at least partially within the barrel, the plunger rod including a stopper. The stopper includes an elastomeric body, a solid lubricant on at least a portion of an exterior of the elastomeric body, and a thin film lubricant positioned between the solid lubricant and an interior of the barrel. The area density of the lubricant on the stopper is about 0.15 μg/cm² to about 50 μg/cm².

According to another Aspect (“Aspect 6”), further to Aspect 5, the thin film lubricant is silicone and the solid lubricant is a fluoropolymer.

According to another Aspect (“Aspect 7”), further to Aspect 5 or Aspect 6, the thin film lubricant reduces an insertion force required to insert the stopper into the barrel by at least about 10%.

According to another Aspect (“Aspect 8”), further to any one of Aspects 5 to 7, the thin film lubricant reduces a breakaway force required to move the stopper in the barrel by at least about 10%.

According to another Aspect (“Aspect 9”), further to any one of Aspects 5 to 8, the thin film lubricant reduces an average glide force required to move the stopper in the barrel by at least about 2%.

According to another Aspect (“Aspect 10”), further to any one of Aspects 5 to 9, an average wet glide force between the stopper and the barrel is less than 5 N.

According to another Aspect (“Aspect 11”), further to any one of Aspects 5 to 10, the mass of the thin film lubricant is present in an amount from about 0.3 μg to about 50 μg.

According to one Aspect (“Aspect 12”), an injection device includes a barrel configured to hold a therapeutic agent and a plunger rod positioned at least partially within the barrel, the plunger rod including a stopper. The stopper includes an elastomeric body, a solid lubricant on at least a portion of an exterior of the elastomeric body, and a thin film lubricant positioned between the solid lubricant and an interior of the barrel. An average number of particles present in the therapeutic agent with a diameter equal to or greater than 10 μm is about 600 or less, and with a diameter equal to or greater than 25 μm is about 60 or less.

According to another Aspect (“Aspect 13”), further to Aspect 12, the thin film lubricant is silicone and the solid lubricant is a fluoropolymer.

According to another Aspect (“Aspect 14”), further to Aspect 12 or Aspect 13, the amount of thin film lubricant present on the stopper is from about 0.3 μg to about 100 μg.

According to one Aspect (“Aspect 15), a method for inserting a stopper into a syringe includes lubricating at least one of the stopper, a vent tube, and a barrel with a thin film lubricant, compressing the stopper such that it has a diameter less than a diameter of the vent tube, inserting the stopper into the vent tube, positioning the vent tube at least partially within the barrel, and inserting the stopper into the barrel. The stopper includes between about 0.3 μg and about 100 μg of thin film lubricant once inserted into the barrel.

According to another Aspect (“Aspect 16”), further to Aspect 15, the thin film lubricant is silicone.

According to another Aspect (“Aspect 17”), further to Aspect 15 or Aspect 16, the step of lubrication reduces an insertion force by at least about 5% during at least one of the inserting steps compared to a method without a lubricating step.

According to another Aspect (“Aspect 18”), further to any one of Aspects 15 to 17, including filling at least a portion of the barrel with a therapeutic agent. The average number of particles present in the therapeutic agent does not exceed about 600 with a diameter equal to or greater than 10 μm, and does not exceed about 60 with a diameter equal to or greater than 25 μm.

According to another Aspect (“Aspect 19”), further to any one of Aspects 15 to 18, lubricating includes lubricating the stopper with the thin film lubricant. The thin film lubricant having a surface area density on the stopper is from about 0.15 μg/cm² to about 50 μg/cm².

According to another Aspect (“Aspect 20”), further to any one of Aspects 15 to 19, inserting the stopper into the barrel requires an insertion force of less than 30 N.

The foregoing Aspects are just that and should not be read to limit or otherwise narrow the scope of any of the inventive concepts otherwise provided by the instant disclosure. While multiple examples are disclosed, still other embodiments will become apparent to those skilled in the art from the following detailed description, which shows and describes illustrative examples. Accordingly, the drawings and detailed description are to be regarded as illustrative in nature rather than restrictive in nature.

### BRIEF DESCRIPTION OF THE DRAWINGS

The accompanying drawings are included to provide a further understanding of the disclosure and are incorporated in and constitute a part of this specification, illustrate embodiments, and together with the description serve to explain the principles of the disclosure.

FIG. 1 is a diagrammatic representation of a syringe in accordance with an embodiment;

FIG. 2 is a cut away view of a stopper within the syringe of FIG. 1 in accordance with an embodiment;

FIG. 3 is a flow diagram for a method of positioning a stopper within a syringe in accordance with an embodiment;

FIGS. 4A and 4B are plots of force displacement data for multiple stopper samples in empty syringes in accordance with Example 2;

FIG. 5 is a plot of breakaway force data for multiple stopper samples in accordance with Example 2;

FIG. 6A is a plot of force displacement data for non-lubricated stopper samples through a vent tube in accordance with Example 3;

FIG. 6B is a plot of force displacement data for lubricated thin film stopper samples through a vent tube in accordance with Example 3;

FIG. 7 is a plot of breakaway force data for multiple stopper and barrel systems in accordance with Example 4;

FIG. 8 is a plot of maximum glide force data for multiple stopper and barrel systems in accordance with Example 4; and

FIGS. 9A-9F are plots of force displacement data for multiple stopper samples in filled syringes in accordance with Example 4.

### DETAILED DESCRIPTION

### Definitions and Terminology

This disclosure is not meant to be read in a restrictive manner. For example, the terminology used in the application should be read broadly in the context of the meaning those in the field would attribute such terminology.

With respect to terminology of inexactitude, the terms “about” and “approximately” may be used, interchangeably. Measurements that are reasonably close to the stated measurement deviate from the stated measurement by a reasonably small amount as understood and readily ascertained by individuals having ordinary skill in the relevant arts. Such deviations may be attributable to measurement error, differences in measurement and/or manufacturing equipment calibration, human error in reading and/or setting measurements, minor adjustments made to optimize performance and/or structural parameters in view of differences in measurements associated with other components, particular implementation scenarios, imprecise adjustment and/or manipulation of objects by a person or machine, and/or the like, for example. In the event it is determined that individuals having ordinary skill in the relevant arts would not readily ascertain values for such reasonably small differences, the terms “about” and “approximately” can be understood to mean plus or minus 10% of the stated value.

### Description of Various Embodiments

Persons skilled in the art will readily appreciate that various aspects of the present disclosure can be realized by any number of methods and apparatus configured to perform the intended functions. It should also be noted that the accompanying drawing figures referred to herein are not necessarily drawn to scale and may be exaggerated to illustrate various aspects of the present disclosure, and in that regard, the figures should not be construed as limiting.

### I. Drug Injection Device

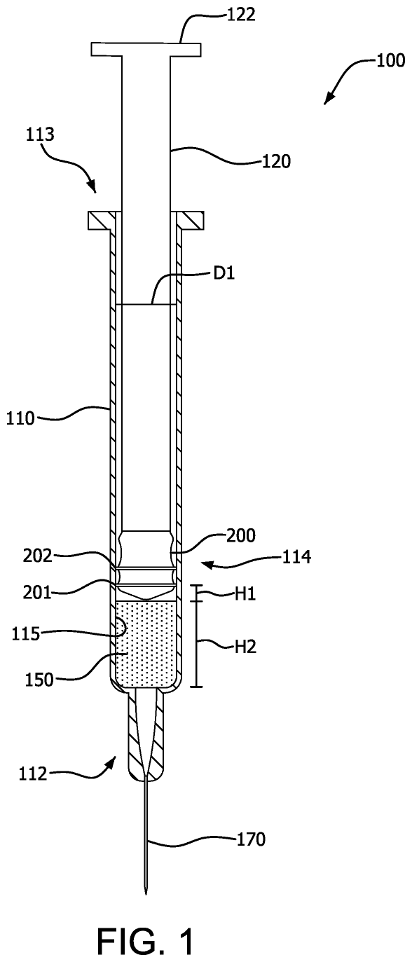

Referring first to FIG. 1, an embodiment of a drug injection device (e.g., a syringe) 100 is provided for delivering at least one therapeutic agent 150 to a patient. Suitable therapeutic agents 150 include, but are not limited to, small molecule drugs, biologics, antibodies, antisense, RNA interference, gene therapy, primary and embryonic stem cells, vaccines, as well as any biologically active compound, and combinations thereof. Other suitable drug injection devices within the scope of the present disclosure includes, for example, auto-injectors.

The illustrative syringe 100 of FIG. 1 includes a barrel 110, which may also be referred to herein for ease of discussion, as a cartridge tube, a plunger rod 120 having a stopper 200, and a piercing element (e.g., needle) 170, each of which is described further below. It is also within the scope of the present disclosure for the syringe 100 to be a “needleless” device having a Luer-Lok™ system (not shown).

The barrel 110 of the syringe 100 contains a liquid therapeutic agent 150 and a distal end 112 that faces toward the patient, a proximal end 113 that faces away from the patient, and an inner surface 115 that faces inward toward the liquid therapeutic agent 150. The barrel 110 may be formed of a hard material, such as a glass material (e.g. borosilicate glass), a ceramic material, one or more polymeric materials (e.g. polypropylene, polyethylene, and copolymers thereof), a metallic material, a plastic material (e.g. cyclic olefin polymers and cyclic olefin copolymers), and combinations thereof. In some embodiments, the barrel 110 may be formed of glass, resin, plastic, or metal with some amount of lubricant present on the inner surface 115 of barrel 110. The barrel 110 may also be supplied with a pre-filled liquid therapeutic agent 150, or alternatively, the therapeutic agent 150 may be drawn into the barrel 110 before use.

The plunger rod 120 of the syringe 100 is movable within the barrel 110 to charge and/or discharge the therapeutic agent 150 by moving the stopper 200 towards the distal end 112 of the barrel 110. The plunger rod 120 includes a head 122 that extends from the proximal end 113 of the plunger rod 120. The stopper 200 is coupled to the opposing end of the plunger rod 120 at the distal end 114 of the plunger rod 120. The illustrative stopper 200 of FIG. 1 contacts the inner surface 115 of the barrel 110 via one or more sealing ribs 201, 202, although any number of sealing ribs and/or non-sealing ribs may be present on the stopper 200. As used herein, the term “sealing rib” means a rib of a stopper that is in contact with the inner surface 115 of the barrel 110 to prevent the passage of air or other contaminants into the barrel 110 or the therapeutic agent 150 from exiting the barrel 110. A “non-sealing” rib as used herein is meant to denote a rib that either does not contact the inner surface 115 or contacts the inner surface 115 of the barrel 110 such that air (or other contaminants) and/or therapeutic agent 150 may pass therethrough.

As shown in FIG. 1, the stopper 200 may be positioned at a predetermined location in the barrel 110 relative to the therapeutic agent 150. The therapeutic agent 150 has a liquid height H2, which depends on the volume of the therapeutic agent 150 in the barrel 110. The stopper 200 may be located at a predetermined stopper height or “headspace” H1 above the therapeutic agent 150, which may be measured from the top surface of the therapeutic agent 150 to the nearest sealing rib 201 of the stopper 200. The headspace H1 may be selected to control the amount of air in the barrel 110 between the stopper 200 and the therapeutic agent 150. In some embodiments, the headspace H1 is less than about 25 mm, less than about 23 mm, less than about 21 mm, less than 1 about 9 mm, less than about 17 mm, less than about 15 mm, less than about 13 mm, less than about 10 mm, less than about 8 mm, less than about 5 mm, less than about 3 mm, less than about 2 mm, less than about 1 mm, or less than about 0.5 mm. The headspace volume may be calculated by multiplying the headspace height H1 by the interior cross-sectional area of the barrel 110, less any volume of the stopper 200 that extends past the sealing rib 201 of the stopper 200 toward the therapeutic agent 150. In some embodiments, it may be advantageous to minimize the headspace H1 to reduce or avoid aggregation of the therapeutic agent 150 in the syringe 100. The stopper 200 is described further in Section II below.

The needle 170 of the syringe 100 as shown in FIG. 1 may be coupled to the distal end 112 of the barrel 110. It is to be appreciated that the depiction of the needle is representative in nature as the syringe 100 may be “needleless” and/or not coupled to the barrel, as may be the case in autoinjector devices. The needle 170 is configured to pierce the patient's skin and inject the therapeutic agent 150 into the patient by pressing the head 122 of the plunger rod 120. In the event that the needle 170 is removable, care should be taken to minimize any bacterial contamination in the barrel 110 when coupling the needle 170 to the barrel 110 and/or when uncoupling the needle 170 from the barrel 110. It is within the scope of the present disclosure for the needle 170 to contain a lubricant for patient comfort without impacting the lubricant exposed to the therapeutic agent 150 in the syringe 100.

### II. Stopper

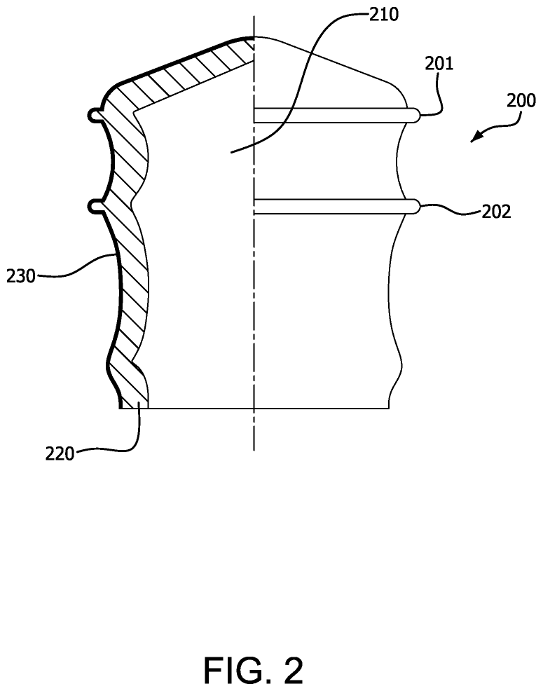

Referring next to FIG. 2, stopper 200 is shown in more detail and includes an elastomeric body 210, a solid lubricant 220 disposed on at least a portion of an outer surface of the elastomeric body 210, and a thin film lubricant 230 disposed between the solid lubricant 220 and the inner surface 115 of the barrel 110. The stopper 200 should have low air and liquid permeability to minimize liquid leakage within the barrel 110 (FIG. 1) and the introduction of air between the stopper 200 and the inner surface 115 of the barrel 110 (FIG. 1) when charging and/or discharging the therapeutic agent 150. In this way, the stopper 200 may resist bacterial contamination in the barrel 110. The stopper 200 should also possess low-friction slidability relative to the barrel 110 to facilitate the charging and/or discharging of the therapeutic agent 150 inside the barrel 110, as described further below.

The elastomeric body 210 of stopper 200 may comprise any elastomer suitable for the application as would be easily identified by one of skill in the art, for example butyl, halobutyl, bromoobutyl, and/or chlorobutyl rubbers, silicone, nitrile, styrene butadiene, polychloropropene, ethylene propylene diene, fluoroelastomers, thermoplastic elastomers (TPE), thermoplastic vulcanizates (TPV), or blends and/or copolymers of any of the foregoing. In other embodiments, the stopper 200 may be constructed of non-elastomeric materials, such as plastics (e.g., polypropylene, polycarbonate, and polyethylene), thermoplastics, or fluoropolymer materials such as ethylene-(perfluoro-ethylene-propene) copolymer (EFEP), polyvinylidene difluoride (PVDF), and perfluoroalkoxy polymer resin (PFA).

The solid lubricant 220 of stopper 200 may also be referred to herein as a coating, a polymer layer, a laminate layer, or a porous layer, and is configured to at least partially cover the surface of elastomeric body 210. In some embodiments, the solid lubricant 220 may be a single layer of a polymer or expanded polymer or the solid lubricant 220 may be a multi-layer construct. The solid lubricant 220 may include a dense inner layer or an open microstructure inner layer to facilitate interaction with the underlying elastomeric body 210, such as receiving the elastomeric body 210 within pores (not shown) of solid lubricant 220. Similarly, the solid lubricant 220 may also include a dense outer layer or an open microstructure outer layer to facilitate interaction of solid lubricant 220 with the thin film lubricant 230, such as receiving the thin film lubricant 230 within pores (not shown) of the solid lubricant 220. The pores may be defined as the spaces between the nodes and fibrils within the microstructure of the solid lubricant 220.

The solid lubricant 220 may be constructed of a fluoropolymer including, but not limited to, polytetrafluoroethylene (PTFE), expanded polytetrafluoroethylene (ePTFE), densified ePTFE, and copolymers and combinations thereof. Other potential fluoropolymers for use as the solid lubricant 220 include, but are not limited to, fluorinated ethylene propylene (FEP), ethylene tetrafluoroethylene (ETFE), polyvinylfluoride, polyvinylidene fluoride (e.g., poly(vinylidene fluoride-co-tetrafluoroethylene) (VDF-co-TFE), poly(vinylidene fluoride-co-trifluoroethylene) (VDE-co-TrFE)), perfluoropropylvinylether, perfluoroalkoxy polymers, and copolymers and combinations thereof, which may be expanded if desired. Yet another potential polymer for use as the solid lubricant 220 includes polyethylene (e.g., expanded ultra-high molecular weight polyethylene (eUHMWPE), such as is described in U.S. Pat. No. 9,926,416 to Sbriglia).

The solid lubricant 220 may have a thickness less than about 30 microns, less than about 25 microns, less than about 20 microns, less than about 15 microns, less than about 10 microns, or less than about 5 microns. In some embodiments, the thickness of solid lubricant 220 may range from about 0.5 microns to about 20 microns, about 1 micron to about 15 microns, about 1 micron to about 10 microns, about 5 microns to about 10 microns, about 1 micron to about 5 microns, or about 7 microns to about 15 microns. The solid lubricant 220 and/or elastomeric body 210 may be pre-treated or post-treated with chemical etching, plasma treating, corona treatment, roughening, or the like to improve the affinity for and bonding of the solid lubricant 220 to elastomeric body 210 and/or thin film lubricant 230 to solid lubricant 220.

The thin film lubricant 230 of the stopper 200 is configured to be positioned between solid lubricant 220 and the inner surface 115 of the barrel 110, and to further reduce friction between the stopper 200 and the barrel 110. In some embodiments, thin film lubricant 230 is provided at a level sufficient to reduce friction in the syringe 100, while minimizing the amount of thin film lubricant 230 that may diffuse into the therapeutic agent 150, and while maintaining a relatively secure seal between stopper 200 and inner surface 115 of the barrel 110. The thin film lubricant 230 may interact with the solid lubricant 220 through chemical and/or physical features of the solid lubricant 220 such that the solid lubricant 220 aids in retaining the thin film lubricant 230 on the stopper 200 instead of diffusing into the therapeutic agent 150. For example, the thin film lubricant 230 may be incorporated onto the nodes and/or the fibrils and/or into the pores (not shown) of the solid lubricant 220. Although the thin film lubricant 230 is shown as a continuous coating layer in FIG. 2, it is within the scope of the present disclosure for thin film lubricant 230 to cover only discrete area(s) of the solid lubricant 220.

In at least one embodiment, the thin film lubricant 230 is coated directly onto the solid lubricant 220 of the stopper 200 by spray coating or by contacting the stopper 200 with another substrate (e.g., a coating tube) that contains an amount of the thin film lubricant 230. The thin film lubricant 230 may also be baked or crosslinked onto the solid lubricant 220 of the stopper 200. Applying the thin film lubricant 230 directly onto the solid lubricant 220 of the stopper 200 may aid in preventing the thin film lubricant 230 from diffusing into the therapeutic agent 150. As further described herein, the thin film lubricant 230 may also be applied to the solid lubricant 220 of the stopper 200 through a vent or insertion tube (not shown) or through the barrel 110. In some embodiments, the thin film lubricant 230 may be applied directly to the barrel 110. Additionally, the thin film lubricant 230 may be applied to a limited number of portions of barrel 110 in order to reduce the overall amount of the thin film lubricant 230 in the system. In one embodiment, the thin film lubricant 230 may be applied only on an upper portion of the barrel 110 that will be in contact with the stopper 200 in an unused configuration, and the thin film lubricant 230 may be absent from a lower portion of barrel 110 that will be in contact with the therapeutic agent 150 and the stopper 200 in a used configuration.

The thin film lubricant 230 may be any solid or liquid lubricant. In some embodiments, the thin film lubricant 230 is silicone oil. In other embodiments, the thin film lubricant 230 may be another lubricant such as polysorbate. Additionally, the thin film lubricant 230 may be chemically or physically altered to improve its affinity for the solid lubricant 220, thereby decreasing the amount of thin film lubricant 230 that may be removed from the stopper 200. In some embodiments, the thin film lubricant is configured to have a greater affinity for the solid lubricant 220 than the barrel 110 and/or the therapeutic agent 150.

The amount of the thin film lubricant 230 that is applied to the stopper 200 may vary. In at least one embodiment, the thin film lubricant 230 is applied at a “low” level, which may be in an amount from about 0.3 μg to about 100 μg per stopper 200. The amount of the thin film lubricant 230 applied to the stopper 200 may be from about 0.3 μg to about 100 μg, from about 5 μg to about 100 μg, from about 0.3 μg to about 90 μg, from about 5 μg to about 90 μg, from about 0.3 μg to about 80 μg, from about 5 μg to about 80 μg, from about 0.3 μg to about 70 μg, from about 5 μg to about 70 μg, from about 0.3 μg to about 60 μg, from about 5 μg to about 60 μg, from about 0.3 μg to about 50 μg, from about 5 μg to about 50 μg, from about 0.3 μg to about 40 μg, from about 5 μg to about 40 μg, from about 0.3 μg to about 30 μg, from about 5 μg to about 30 μg, from about 0.3 μg to about 20 μg, from about 5 μg to about 20 μg, from about 0.3 μg to about 10 μg, from about 5 μg to about 10 μg. In terms of surface density, for a 1 mL stopper 200 having a surface area of 2 cm², thin film lubricant 230 may be present on stopper 200 in an amount from about 0.15 μg/cm² to about 50 μg/cm², from about 2.5 μg/cm² to about 50 μg/cm² from about 0.15 μg/cm² to about 45 g/cm², from about 2.5 μg/cm² to about 45 μg/cm², from about 0.15 μg/cm² to about 40 μg/cm², from about 2.5 μg/cm² to about 40 μg/cm², from about 0.15 μg/cm² to about 35 μg/cm², from about 2.5 μg/cm² to about 35 μg/cm² from about 0.15 μg/cm² to about 30 μg/cm², from about 2.5 μg/cm² to about 30 μg/cm², from about 0.15 μg/cm² to about 25 μg/cm², from about 2.5 μg/cm² to about 25 μg/cm², from about 0.15 μg/cm² to about 20 μg/cm², from about 2.5 μg/cm² to about 20 μg/cm², from about 0.15 μg/cm² to about 15 μg/cm², from about 2.5 μg/cm² to about 15 μg/cm², from about 0.15 μg/cm² to about 10 μg/cm², from about 2.5 μg/cm² to about 10 μg/cm², from about 0.15 μg/cm² to about 5 μg/cm², from about 2.5 μg/cm² to about 5 μg/cm². Of course, the surface density may vary based on the size of the stopper 200.

The small amount of the thin film lubricant 230 applied to the solid lubricant 220 of the stopper 200 and the interaction between the thin film lubricant 230 and the solid lubricant 220 may prevent particles of the thin film lubricant 230 from entering the therapeutic agent 150 when the syringe 100 is stored or in use. This “small” amount of the thin film lubricant 230 can be measured using gas chromatography (GC) mass spectrometry, inductively coupled plasma (ICP) mass spectrometry, and/or by the amount of particles present in the barrel 110 that are measured in water for injection (WFI) after the WFI has been exposed to a fully assembled syringe 100 (e.g., a glass barrel 110, a stopper 220, and at least one therapeutic agent 150). For example, the average number of particles present in the therapeutic agent 150 may not exceed about 600 particles/mL with diameters greater than 10 μm, and about 60 particles/mL with diameters greater than 25 μm.

The stopper 200 may be coated with the thin film lubricant 230 at a low level, as described above, such that the small amount of the thin film lubricant 230 that diffuses into the therapeutic agent 150 within the syringe 100 or otherwise separates from the stopper 200 is reduced or minimized. The interaction between the thin film lubricant 230 and the solid lubricant 220 may also contribute to retaining the thin film lubricant 230 on the stopper 200.

The thin film lubricant 230 may allow for a lower average insertion force of the stopper 200 into the barrel 110 when compared to stoppers without a thin film lubricant. In some embodiments, the presence of the thin film lubricant 230 may decrease the average insertion force by about 10%, about 15%, about 20%, about 25%, about 30%, or more when compared to stoppers without a thin film lubricant. In some embodiments, the insertion force (i.e., the force required to insert the stopper into the barrel) may be less than 35 N, less than 30 N, less than 25 N, less than 20 N, or within any range including any two of these values as endpoints. In at least one embodiment, the insertion force of inserting the stopper 200 into the barrel 110 through a vent tube may be about 20 N to about 30 N for a 1 ml syringe 100.

The thin film lubricant 230 may allow for a lower breakaway force of the stopper 200 in the barrel 110 when compared to stoppers without a thin film lubricant. In certain embodiments, the presence of the thin film lubricant 230 may decrease the breakaway force (wet or dry) by about 10%, about 15%, about 20%, about 25%, or more compared to stoppers without a thin film lubricant. In some embodiments, the dry breakaway force (i.e., the force required to initially move a stopper in a barrel without any liquid in the barrel) between the stopper 200 and the barrel 110 may be less than about 15 N, less than about 12.5 N, less than about 10 N, less than about 7.5 N, or less than about 5 N. In some embodiments, the dry breakaway force between the stopper 200 and the barrel 110 may be from about 8 N to about 12 N for a 1 ml syringe 100. Furthermore, in some embodiments the wet breakaway force (i.e., the force required to initially move a stopper in a barrel with liquid in the barrel) may be less than about 15 N, less than about 10 N, less than about 7 N, less than about 5 N, or within any range including any two of these values as endpoints. In at least one embodiment, the wet breakaway force between the stopper 200 and the barrel 110 may be about 7 N to about 10 N.

The following table sets forth estimated wet and dry average breakaway force values for stoppers without a thin film lubricant (“No Lubricant”) compared to otherwise equivalent stoppers 200 having the thin film lubricant 230 as described herein (“Low Lubricant”).

TABLE 1
Estimated Breakaway Forces for Varying Stopper Sizes

| | Stopper | Wet Breakaway Force | | Dry Breakaway Force | |
|---|---|---|---|---|---|
| Syringe | Surface | (N) | | (N) | |
| Volume | Area | No | Low | No | Low |
| (mL) | (cm²) | Lubricant | Lubricant | Lubricant | Lubricant |
| 1 | 2.01 | 4-15 | 3-13 | 11-13.5 | 8-12 |
| 2.25 | 2.93 | 4-7 | 3-6 | 12.5-14 | 10-12 |
| 0.5 | 1.10 | 2.5-4.5 | 2-4 | 3-5.5 | 2-5 |

The thin film lubricant 230 may also allow for a lower average glide force of the stopper 200 in the barrel 110 when compared to comparable stoppers without a thin film lubricant. In some embodiments, the presence of the thin film lubricant 230 may decrease the average slide force by about 2%, about 5%, about 10%, about 15%, about 20%, about 25%, or more compared to stoppers without a thin film lubricant 230. In some embodiments, the disclosed stopper 200 may demonstrate an equivalent glide force compared to stoppers without a thin film lubricant, but lower insertion and/or breakaway forces.

In some embodiments, the wet max glide force (i.e., the maximum force required to move a stopper once the stopper has begun moving in a barrel with liquid in the barrel) and/or the wet average glide force (i.e., the average force required to move a stopper once the stopper has begun moving in a barrel with liquid in the barrel) may be less than 10 N, less than 9 N, less than 8 N, less than 7 N, less than 6 N, less than 5 N, less than 4 N, less than 3 N, or within any range including any two of these values as endpoints. For example, the wet average glide force between the stopper 200 and the barrel 110 may be about 3 N to about 5 N for a 1 ml syringe 100. The wet max glide force between the stopper 200 and the barrel 110 may be about 5 N to about 8 N for a 1 mL syringe 100.

Additionally, the stopper 200 may also demonstrate an improved or equivalent seal between the stopper 200 and the inner surface 115 of the barrel 110 when compared to stoppers without a thin film lubricant. In some embodiments, this seal may be evaluated using container closure integrity test methods, as described further below.

### III. Assembly Method

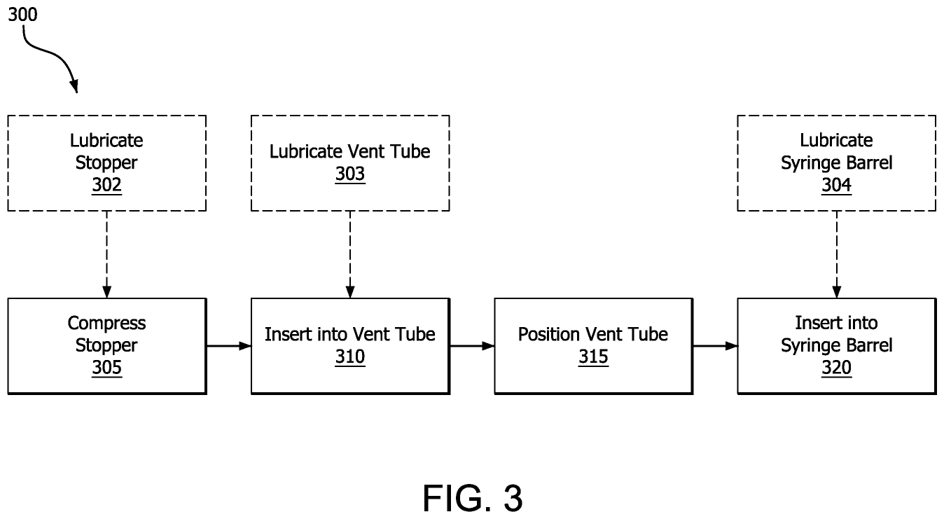

Referring now to FIG. 3, a method 300 for inserting the stopper 200 into the barrel 110 is shown. Method 300 includes: a compressing step 305 that involves compressing the stopper 200, a first inserting step 310 that involves inserting the stopper 200 into a vent tube or an insertion tube (not shown in the FIGS.), a positioning step 315 that involves positioning the vent tube in the barrel 110, and a second inserting step 320 that involves inserting the stopper 200 into the barrel 110. Method 300 also contains one or more lubricating steps, including a lubricating step 302 that involves lubricating the stopper 200 with the thin film lubricant 230, a lubricating step 303 that involves lubricating the vent tube with thin film lubricant 230, and/or a lubricating step 304 that involves lubricating the barrel 110 with the thin film lubricant 230. The stopper 200 is first compressed such that it may fit within the vent tube during the compressing step 305. The stopper 200 may optionally be lubricated during the lubricating step 302 or otherwise coated with the thin film lubricant 230 before the compressing step 305.

The compressed stopper 200 may then be inserted into the vent tube during the first inserting step 310. The vent tube has a diameter that is smaller than the inner diameter D1 of barrel 110 (see FIG. 1). The vent tube may also be optionally lubricated during the lubricating step 303 with the thin film lubricant 230 before the stopper 200 is inserted into the vent tube during the first inserting step 310. In embodiments where the vent tube is lubricated with the thin film lubricant 230, the stopper 200 may not be lubricated before insertion into the vent tube and may be coated with the thin film lubricant 230 through contact with the thin film lubricant 230 on an interior surface of the vent tube. Once the stopper 200 is inserted into the vent tube, the vent tube is then positioned within the barrel 110 during the positioning step 315. The interior of the barrel 110 may also optionally be lubricated during the lubricating step 304 with the thin film lubricant 230 either instead of or in addition to lubricating the stopper 200 during the lubricating step 302 and/or lubricating the vent tube during the lubricating step 303. The stopper 200 may then be inserted into the barrel 110 during the second inserting step 320. Moving stopper 200 from the vent tube into barrel 110 may be completed with the use of an insertion rod. Further examples of insertion systems and methods are disclosed in PCT Publication No. WO 2020/112612 to W. L. Gore & Associates. In some embodiments, the stopper 200 may be inserted into the barrel 110 through vacuum insertion, with a vent tube, or combinations thereof.

Persons skilled in the art will readily appreciate that various aspects of the present disclosure can be realized by any number of methods and apparatuses configured to perform the intended functions. It should also be noted that the accompanying drawings and/or figures referred to herein are not necessarily drawn to scale but may be exaggerated to illustrate various aspects of the present disclosure, and in that regard, the drawings and/or figures should not be construed as limiting.

The syringe and stopper depicted in FIGS. 1 and 2 are provided as an example of the various features of the syringe and, although the combination of illustrated features is considered to be clearly within the scope of invention, that example and its illustration is not meant to suggest the inventive concepts provided herein are limited from fewer features, additional features, or alternative features to one or more of those features shown in FIGS. 1 and 2.

### Test Methods

It should be understood that although certain methods and equipment are described below, other methods or equipment determined suitable by one of ordinary skill in the art may be alternatively utilized.

### Quantification of Lubricant (Silicone)

For determining the quantity of silicone present on a stopper, stoppers were extracted (i.e. reflux extraction or sonication at elevated temperatures) with a suitable organic solvent, in this case n-heptane. The determination of totally extractable silicone was then made with Graphite Furnace Atomic Absorption Spectrometry (GF-AAS) using the standard addition method. Organically soluble silicon (Si) was measured by GF-AAS and calculated as polydimethylsiloxane (PDMS) with a Si-content of 38 wt.-%. The data for multiple stoppers were averaged for each type of stopper tested.

### Insertion Force Method

Insertion force was measured with an Instron UTM (Compression test, pre-Load of 0.02 N at 20 mm/min, test speed of 40 mm/s, end of test criteria of 70 N, data acquisition rate of 1 ms). Six of each plunger type (lubricated and non-lubricated) were tested for insertion force by insertion into a barrel through a vent tube.

### Breaking and Sliding Friction Tests

Breakaway force and average slide force were measured by inserting a stopper using a vent tube stopper insertion machine into an empty syringe (for “dry” measurements) or a syringe filled with 0.96 mL of Water for Injectables (WFI) (for “wet” measurements). The syringe used was a staked needle design with a 29 gauge ½ inch needle. An appropriate plunger rod to match the stopper was fitted into the assembled syringe system without moving or disturbing the stopper. The system was placed into a holder on a force displacement analyzer, and the test speed of 250 mm/minute was established, after which force displacement data was obtained. The maximum force obtained was recorded. The force displacement instrument used was a TA XT Plus Texture Analyzer with a TA 270N syringe test fixture (Hamilton, Mass.) or a Zwick UTM.

### Container Closure Integrity Testing

Each syringe was filled with WFI to a nominal value. The WFI in the syringe contained no trace of blue (solution or coloring). The stopper to be examined was fitted into the syringe. The syringe was then immersed in a solution of 0.1% (1 g per L) methylene blue, and external pressure was reduced to 27 kPa for 10 minutes using a Haug Pack Vac Chamber/System, in accordance with USP 1207. Pressure was then returned to atmospheric, and the syringe was left submerged for 30 minutes. The outside of the vial was then rinsed. The stopper was considered to pass the test if the syringe contained no trace of blue solution.

### Subvisible Particle Characterization

Subvisible particles, including silicone, were characterized through Micro-Flow Imaging (MFI). Syringes barrels were filled with WFI and a stopper was inserted as previously described. Samples were inverted 20 times and the ejected WFI was analyzed utilizing a FlowCAM VS-1 Microflow Imaging Machine with a 10× Objective Lens, 10× Collimator, and FC100x2 100 μm Flow Cell for 10λ.

### EXAMPLES

### Example 1: Preparing Stoppers

1 mL Long syringe stoppers were used for all the following examples. Each stopper was tested using silicone oil as the thin film lubricant. The tested stoppers are classified as follows: The control 1 and control 2 stoppers did not have silicone added, however as shown in Table 2 below, a trace amount of silicone was found on the control 2 stopper. The “SA” stopper was coated with a thin film of silicone by contacting the stopper with other silicone coated stoppers and agitating the stoppers. The “SV” stopper was coated with a thin film of silicone by lubrication of the vent tube with which the stopper was inserted into its respective barrel. Both the SA and SV stoppers were inserted with a vent tube from Supplier 1, but the SV vent tube had been treated with a trace amount of silicone prior to insertion. The “SVT” stopper was lubricated with silicone in a similar manner to SV, but with a vent tube from Supplier 2. The stainless-steel tubes form both suppliers were prepared generally according to the teachings of U.S. Pat. No. 10,369,292 to LaRose. The amount of silicone present on each of the stoppers was determined using the procedure outlined above, and the results are tabulated in Table 2 below. The limit of quantification for this procedure is 0.6 μg/stopper for Control 1 and 1.3 μg/stopper for the remainder of the stoppers, and uncertainties were calculated with k=2.

TABLE 2
Average Amount of Silicone on Each Type of Stopper

| | Amount of Extractable Silicone |
|---|---|
| Stopper | [μg/stopper] |
| Control 1 | <0.6 |
| Control 2 | 2.5 ± 1.0 |
| SA | 8.7 ± 2.6 |
| SV | 4.7 ± 1.4 |
| STV | 2.3 ± 0.9 |

Since the procedure for determining the amount of silicone on the stoppers is destructive, the same stoppers were not used for the following example test as the ones used in determining the amount of silicone present. Instead, similar stoppers were used which were prepared by the same methods and accordingly should contain similar levels of silicone. The control stoppers used in the following examples were closest to Control 2 and may be referred to simply as “control” below.

### Example 2: Container Closure Integrity and Dry Sliding Performance

20 syringes incorporating stoppers with thin film lubricant were tested for container closure integrity. No samples showed evidence of blue dye ingress.

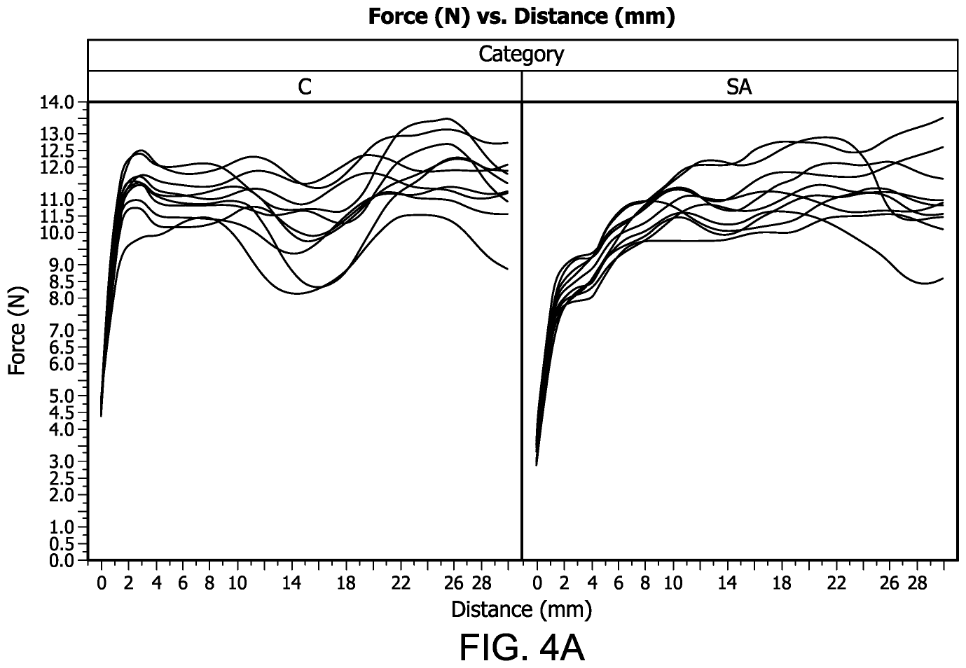

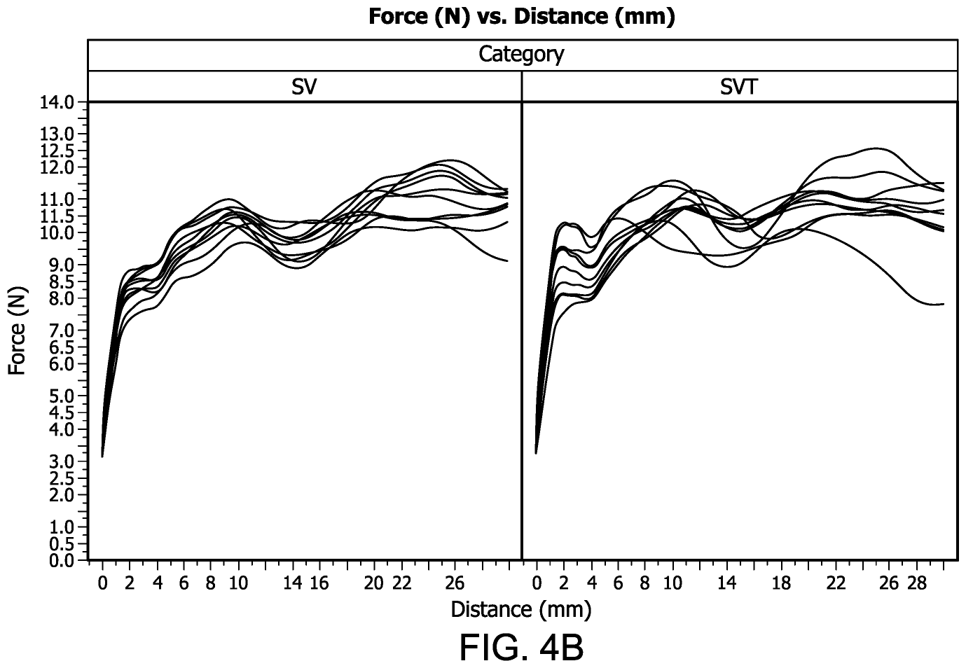

Referring now to FIG. 4, the force displacement data for each stopper sample inserted into an empty bare glass syringe barrel (no solid or liquid lubricant thereon) is plotted. From this data, the breakaway force and the average glide force were determined. As shown, the stoppers coated with a thin film lubricant displayed more consistent slide force profiles as compared to the non-lubricated control.

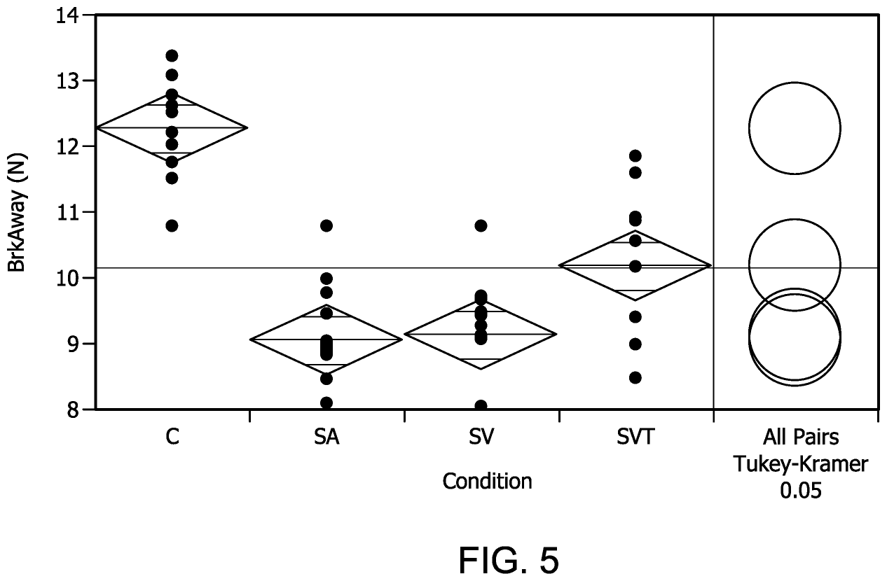

Referring next to FIG. 5, the dry breakaway force for each stopper sample is plotted. As shown, each of the stoppers coated with a thin film lubricant demonstrated significantly lower dry breakaway force when compared to the non-lubricated control. This decrease indicates that, while the levels of silicone present on the stoppers is low, the thin film lubricant still provides a benefit to the force required to initiate and continue movement of the stopper.

### Example 3: Insertion Force

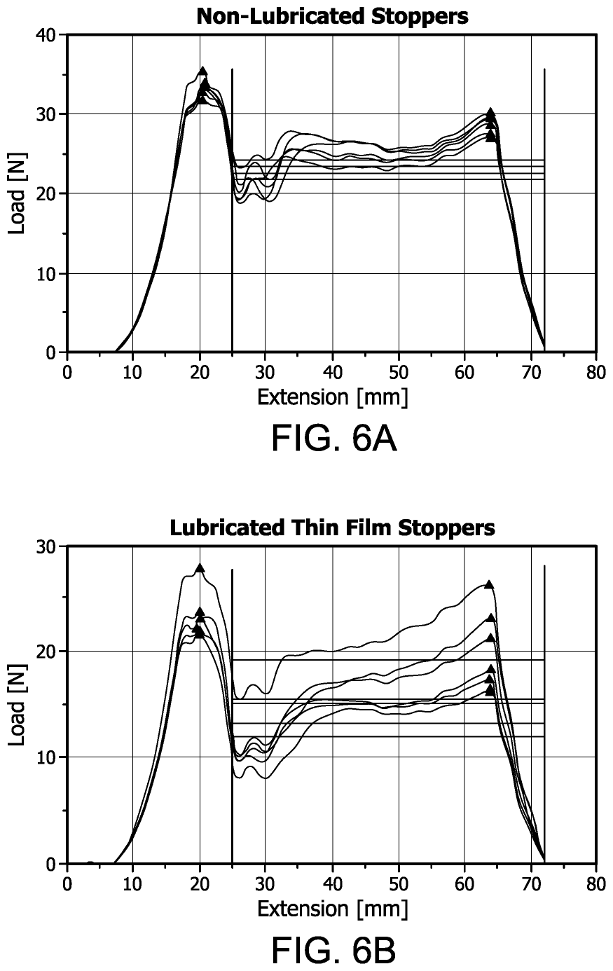

For this example, stoppers with and without a thin film lubricant (i.e., silicone oil) were inserted into a barrel via a vent tube, and the insertion force was measured. The stoppers tested were GORE ImproJect 1 mL Long. The stopper samples with low silicone levels from presence of the thin film lubricant were prepared by placing the stoppers (n=108) into a small container and adding 1 L of Dow Corning 360 silicone oil into the container. The container was tumbled until all stoppers were evenly coated. A “generic” stopper (available from West FluroTec®) was intended to be tested as a comparative example. However, the generic stoppers exceeded the maximum insertion force of the instrument and were not able to be inserted into a barrel using the same vent tube (having the same vent tube geometry). The results are summarized in Table 3 below and the corresponding force displacement data through a vent tube is shown in FIG. 6.

TABLE 3
Insertion Force Results

| Stopper | Average (N) | Min (N) | Max (N) | St dev (N) |
|---|---|---|---|---|
| Non-Lubricated | 33.49 | 31.81 | 35.49 | 1.208 |
| Lubricated with | 23.33 | 21.56 | 27.80 | 2.326 |
| Thin Film | | | | |
| Generic | N/A | N/A | N/A | N/A |

As shown in Table 3 above, the addition of a thin film lubricant significantly reduced the insertion force required to insert the stopper into a barrel with a vent tube.

### Example 4: Wet Breakaway and Glide Force Testing

In this example, a number of stoppers and barrels were tested for breakaway force and glide force. Stoppers were either non-lubricated, lubricated with a thin film lubricant, or “generic” stoppers (Non-lubricated or lubricated plungers were GORE ImproJect 1 mL Long. Generic plungers were West FluroTec® with B2-coating). The stoppers were inserted into a non-lubricated barrel or a lubricated barrel (barrels were Schott 1 mL long barrels siliconized by using the ZebraSci Flex FP Spray System and Dow Corning 360 silicone oil). The stopper and barrel systems were labelled as indicated in Table 4 below. The thin-film lubricant applied was silicone oil, thus stoppers and/or barrels comprising a thin film lubricant may also be referred to hereafter as “siliconized”.

TABLE 4
Sample Labels of Stopper and Barrel Systems

| Sample | Barrel | Stopper |
|---|---|---|
| A | Non-siliconized | Non-siliconized |
| B | Non-siliconized | Low Siliconized |
| C | Non-siliconized | Generic |
| D | Low Siliconized | Non-siliconized |
| E | Low Siliconized | Low Siliconized |
| F | Low Siliconized | Generic |

The samples for breakaway force (or break loose force) and glide force testing were prepared using the manual vent tube insertion fixture for the Gore stoppers, and a Groninger Mechanical Stoppering Machine for the West Fluorotec stoppers. Each sample was filled with 1 ml of water for injection (WFI). Six of each configuration (A-F) were tested for wet breakaway force and glide force.

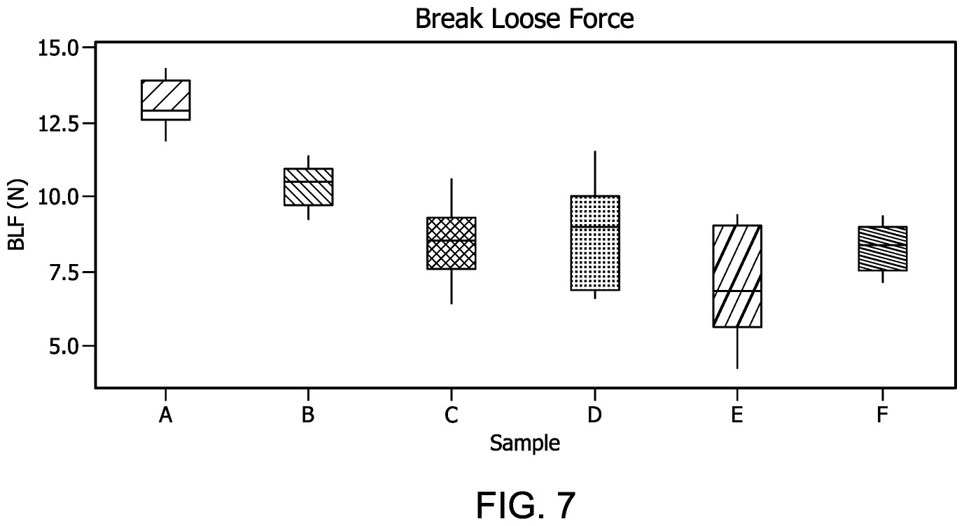

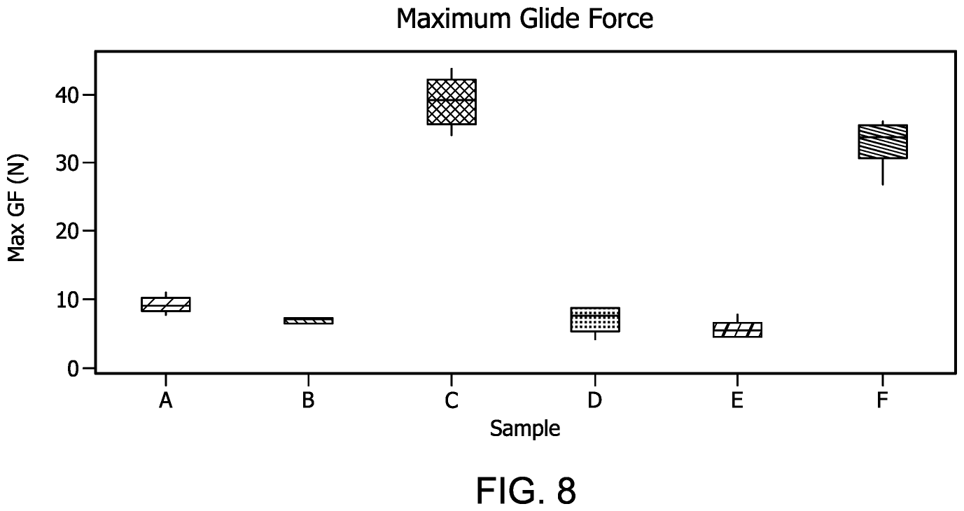

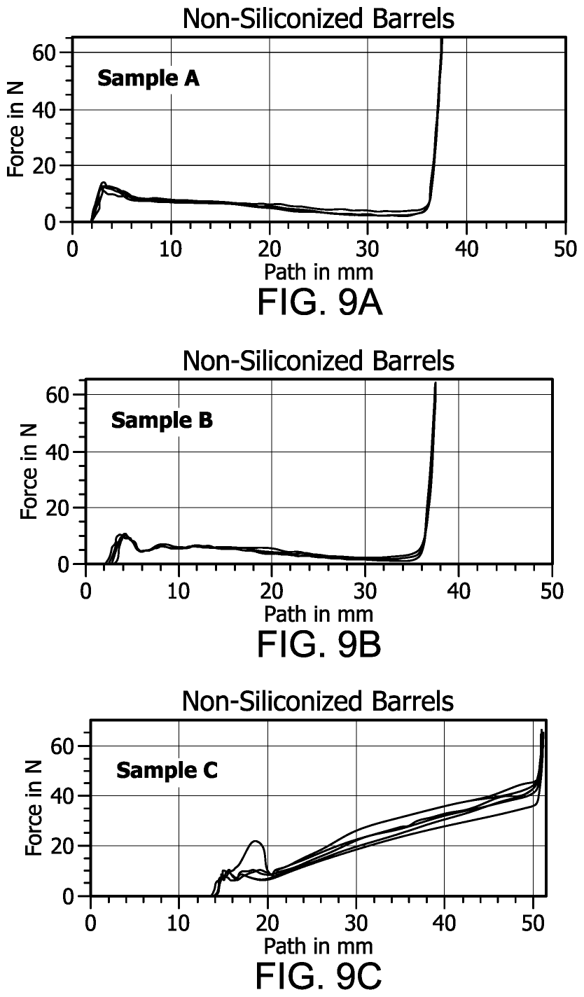

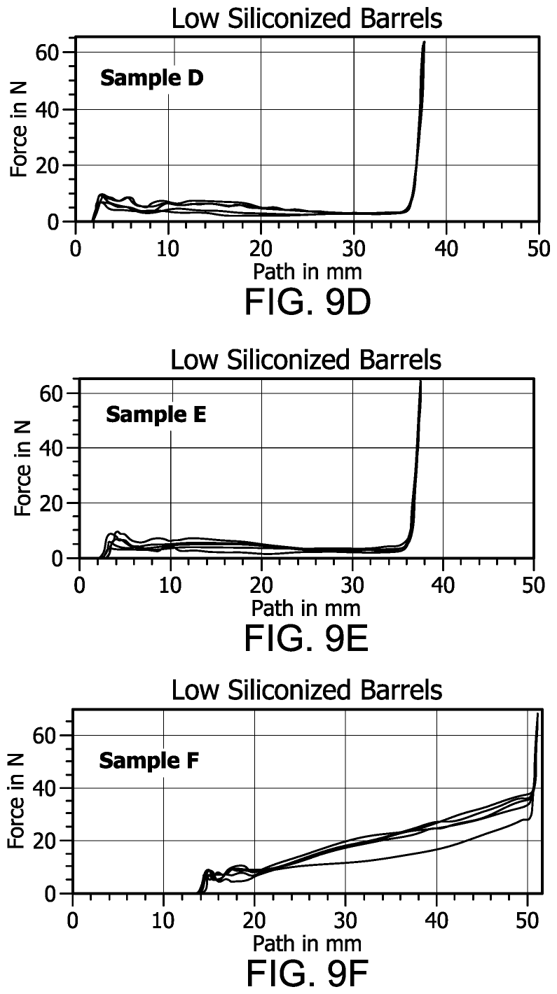

The wet breakaway force data are summarized in FIG. 7 and Table 5 below. The wet glide force data are summarized in FIG. 8 and Tables 6 and 7 below. FIG. 8 and Table 6 show the maximum glide force, while Table 7 shows the average glide force. FIG. 9 shows the corresponding slide curves for each group of samples.

TABLE 5
Wet Breakaway Force Results

| Sample | Average (N) | Min (N) | Max (N) | St dev (N) |
|---|---|---|---|---|
| A | 13.13 | 11.92 | 14.31 | 0.825 |
| B | 10.41 | 9.22 | 11.39 | 0.758 |
| C | 8.48 | 6.38 | 10.59 | 1.363 |
| D | 8.77 | 6.55 | 11.57 | 1.855 |
| E | 7.04 | 4.17 | 9.38 | 1.908 |
| F | 8.30 | 7.15 | 9.34 | 0.837 |

TABLE 6
Wet Maximum Glide Force Results

| Sample | Average (N) | Min (N) | Max (N) | St dev (N) |
|---|---|---|---|---|
| A | 9.21 | 7.72 | 10.94 | 1.178 |
| B | 6.90 | 6.33 | 7.34 | 0.390 |
| C | 39.05 | 34.17 | 43.78 | 3.492 |
| D | 7.09 | 4.20 | 9.03 | 1.912 |
| E | 5.60 | 4.30 | 7.46 | 1.197 |
| F | 32.98 | 26.65 | 36.25 | 3.403 |

TABLE 7
Wet Average Glide Force Results

| Sample | Average (N) | Min (N) | Max (N) | St dev (N) |
|---|---|---|---|---|
| A | 5.17 | 4.81 | 6.07 | 0.475 |
| B | 4.43 | 4.09 | 4.89 | 0.347 |
| C | 22.28 | 19.14 | 26.63 | 2.760 |
| D | 4.20 | 2.62 | 5.19 | 1.080 |
| E | 3.81 | 2.56 | 4.80 | 0.756 |
| F | 18.29 | 13.51 | 19.88 | 2.432 |

As shown in FIGS. 7 to 9 and Tables 5 to 7, the application of a thin film lubricant (such as silicone oil) significantly lowered the breakaway force and glide force of a stopper and barrel system. Regarding breakaway force, the non-siliconized barrel with the non-siliconized stopper had the highest average breakaway force, followed by the non-siliconized barrel with the low siliconized stopper, and then by the non-siliconized barrel with the generic plunger. Regarding glide force, the highest glide force was observed in the non-siliconized barrel with the generic stopper, and the low-siliconized barrel with the generic stopper. The non-siliconized barrel and non-siliconized stopper showed the next highest maximum glide force compared to the remaining samples. Application of a “low” level of a thin film lubricant reduced breakaway and/or glide force while also minimizing the amount of lubricant in the system.

### Example 5—Silicone Quantification for Examples 3 and 4

The amount of silicone present on each of the stoppers and barrels in Examples 3 and 4 was determined using the procedure outlined above, and the results were calculated and are set forth in Tables 8 and 9, respectively. The limit of quantification of silicone for this example was 0.3 μg/stopper and 0.5 μg/barrel.

TABLE 8
Mean Total Silicone on Each Type of Stopper

| | Amount of Extractable Silicone |
|---|---|
| Stopper | [μg/stopper] |
| Non-siliconized Gore Stopper | <0.30 |
| Low Siliconized Gore Stopper | 10.8 |
| West FluroTec Stopper | 41 |

TABLE 9
Mean Total Silicone on Each Type of Barrel

| | Amount of Extractable Silicone |
|---|---|
| Barrel | [μg/barrel] |
| Non-siliconized Barrel | <0.50 |
| Low Siliconized Barrel | 6.5 |

### Example 6—Subvisible Particle Characterization

Syringes incorporating stoppers with the thin film lubricant (Sample B) were tested for particles by MFI as described above. Results shown in Table 10 are the average of 3 replicate runs. Silicone particles were identified via image analysis.

TABLE 10
Average Particle Count for Sample B

| | |
|---|---|
| All Particles | Silicone Only Particles |

| | | | | | |
|---|---|---|---|---|---|
| 10-25 | μm | 4.17 | 10-25 | μm | 0 |
| >25 | μm | 0.00 | >25 | μm | 0 |

The invention of this application has been described above both generically and with regard to specific embodiments. It will be apparent to those skilled in the art that various modifications and variations can be made in the embodiments without departing from the scope of the disclosure. Thus, it is intended that the embodiments cover the modifications and variations of this invention provided they come within the scope of the appended claims and their equivalents.

1. A syringe comprising:
a barrel configured to hold a therapeutic agent;
a plunger rod positioned at least partially within the barrel, the plunger rod including a stopper, the stopper comprising:
an elastomeric body;
a solid lubricant on at least a portion of an exterior of the elastomeric body; and
a thin film lubricant positioned between the solid lubricant and an interior of the barrel, wherein a mass of the thin film lubricant on the stopper is from about 0.3 μg to about 100 μg.

2. The syringe of claim 1, wherein the thin film lubricant is silicone and the solid lubricant is a fluoropolymer.

3. The syringe of claim 1, wherein the solid lubricant and the thin film lubricant on the stopper represent the total lubricant in the syringe.

4. The syringe of claim 1, wherein the stopper is configured to be slidably moved within the barrel with a dry breakaway force less than about 15 N.

5. A syringe comprising:
a barrel configured to hold a therapeutic agent;
a plunger rod positioned at least partially within the barrel, the plunger rod including a stopper, the stopper comprising:
an elastomeric body;
a solid lubricant on at least a portion of an exterior of the elastomeric body; and
a thin film lubricant positioned between the solid lubricant and an interior of the barrel, wherein an area density of the lubricant on the stopper is about 0.15 μg/cm² to about 50 μg/cm².

6. The syringe of claim 5, wherein the thin film lubricant is silicone and the solid lubricant is a fluoropolymer.

7. The syringe of claim 5, wherein the thin film lubricant is configured to reduce an insertion force required to insert the stopper into the barrel by at least about 10%.

8. The syringe of claim 5, wherein the thin film lubricant is configured to reduce a breakaway force required to move the stopper in the barrel by at least about 10%.

9. The syringe of claim 5, wherein the thin film lubricant is configured to reduce an average glide force required to move the stopper in the barrel by at least about 2%.

10. The syringe of claim 5, wherein an average wet glide force between the stopper and the barrel is less than 5 N.

11. The syringe of claim 5, wherein the mass of the thin film lubricant is present on the stopper in an amount from about 0.3 μg to about 50 μg.

12. An injection device comprising:
a barrel configured to hold a therapeutic agent;
a plunger rod positioned at least partially within the barrel, the plunger rod including a stopper, the stopper comprising:
an elastomeric body;
a solid lubricant on at least a portion of an exterior of the elastomeric body; and
a thin film lubricant positioned between the solid lubricant and an interior of the barrel, wherein an average number of particles present in the therapeutic agent with a diameter equal to or greater than 10 μm is about 600 or less, and with a diameter equal to or greater than 25 μm is about 60 or less.

13. The syringe of claim 12, wherein the thin film lubricant is silicone and the solid lubricant is a fluoropolymer.

14. The syringe of claim 13, wherein the amount of thin film lubricant present on the stopper is from about 0.3 μg to about 100 μg.

15-20. (canceled)
